(19) United States
(12) Patent Application Publication
(10) Pub. No.: US 2005/0014816 A1
(43) Pub. Date: Jan. 20, 2005

(54) Thiophene amino acid derivatives, process for preparing them and pharmaceutical compositions containing them

(75) Inventors: Delphine Compere, Paris (FR); Anne-Claude Dublanchet, Antony (FR); Karine Courte, Issy-les-Moulineaux (FR); Stephane Blais, Palaiseau (FR); Pierre Ducrot, Verrieres le Buisson (FR); Philippe Cluzeau, Loisin (FR); Alexis Denis, Paris (FR)

Correspondence Address: PFIZER INC., PATENT DEPARTMENT, MS8260-1611, EASTERN POINT ROAD, GROTON, CT, 06340, US

(73) Assignee: Pfizer Inc

(21) Appl. No.: 10/884,059

(22) Filed: Jul. 2, 2004

Related U.S. Application Data
(60) Provisional application No. 60/494,654, filed on Aug. 12, 2003.

(30) Foreign Application Priority Data
Jul. 3, 2003 (EP) 03291641.3

(51) Int. Cl. A61K 31/381; C07D 333/34; C 07D 4 9/02

(52) U.S. Cl. 514/444; 514/448; 549/64

(57) ABSTRACT

The invention provides compounds of Formula (I), 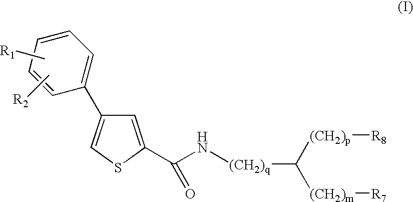
stereoisomers thereof, or pharmaceutically acceptable salts of said compounds or stereoisomers, wherein R₁, R₂, m, p, q, R₇ and R₈ are as defined below, as well as compositions comprising the same, processes for making the same, and methods of using the same to treat a variety of diseases, including, those requiring interaction with metalloproteases, and more specifically with macrophage metalloelastase (MMP-12), and for the prevention and treatment of respiratory pathologies such as chronic obstructive bronchopneumopathy (COPD), emphysema, chronic bronchitis, chronic pulmonary inflammation, asthma, cystic fibrosis, acute respiratory distress syndrome (ARDS), respiratory allergies including allergic rhinitis, and also diseases associated with the production of TNFα including severe fibrotic pulmonary disease, pulmonary sarcoidosis and silicosis. The compounds of the present invention also show inhibitory activity on metalloprotease-13 (MMP-13), making them useful for the treatment of pathologies involving this enzyme, such as cancer, osteoporosis, osteoarthritis, arthritis, rheumatoid arthritis, atherosclerosis, multiple sclerosis and cardiac insufficiency.

### FIELD OF THE INVENTION

The present invention relates to novel thiophene amino acid derivatives, for interacting with metalloproteases, and more specifically with macrophage metalloelastase (MMP-12), and for the prevention and treatment of respiratory pathologies such as chronic obstructive bronchopneumopathy (COPD), emphysema, chronic bronchitis, chronic pulmonary inflammation, asthma, cystic fibrosis, acute respiratory distress syndrome (ARDS), respiratory allergies including allergic rhinitis, and also diseases associated with the production of TNFα including severe fibrotic pulmonary disease, pulmonary sarcoidosis and silicosis. The compounds of the present invention also show inhibitory activity on metalloprotease-13 (MMP-13), making them useful for the treatment of pathologies involving this enzyme, such as cancer, osteoporosis, osteoarthritis, arthritis, rheumatoid arthritis, atherosclerosis, multiple sclerosis and cardiac insufficiency. In addition, the invention relates-to methods and processes concerning the same, and to compositions containing the same.

### BACKGROUND OF THE INVENTION

The compounds of the present invention also show, to a lesser extent, inhibitory activity on metalloprotease-13 (MMP-13), making them potentially useful for the treatment of pathologies involving this enzyme, such as cancer, osteoporosis, osteoarthritis, arthritis, rheumatoid arthritis, atherosclerosis, multiple sclerosis and cardiac insufficiency.

Metalloproteases (MMPs) are a large family of proteases that degrade the extracellular matrix and are secreted especially by mesenchymal cells, macrophages and polymorphonuclear leukocytes. Metalloproteases are classified into several subfamilies depending on their primary structure and their specificity. These families especially include collagenases (MMP-1, MMP-8 and MMP-13), stromelysins (MMP-3 and MMP-10), gelatinases (MMP-2 and MMP-9), matrilysin (MMP-7), macrophage metalloelastase (MMP-12) and also MMPs of membrane-bound type (MMP-14, MMP-15, MMP-16 and MMP-17).

MMPs are zinc metalloproteases that have the ability to degrade virtually all the components of the extracellular matrix, ie the interstitium and the basal membranes. Increased synthesis of these enzymes is found in many destructive diseases (inflammatory arthritis, atherosclerosis, tumoral invasion and angiogenesis). MMPs (in particular those with powerful elastolytic activity) are involved in the physiopathology of asthma and chronic obstructive bronchopneumopathies including tobacco-related pulmonary emphysema (COPD).

Human macrophage elastase (HME or MMP-12) shows all the characteristics of the other MMPs. It degrades many macromolecules of the extracellular matrix (gelatin, fibronectin and laminin) and especially elastin. MMP-12 is not synthesized by the circulating monocytes but solely by macrophages or monocytes differentiated in vitro into macrophages. The pathology of emphysema is characterized by destruction of the elastin present in the walls of the pulmonary alveolae. Demonstration of the increase in the level of MMP-12 during the manifestation of this pathology thus suggests a predominant role of this enzyme in the occurrence and development of this disease. Similarly, studies have demonstrated the absence of development of emphysema in MMP-12-deficient mice, these mice being exposed for a long time to cigarette smoke (Science 1997, 277, 2002-2004). More recently, also using MMP-12-deficient mice, in an asthma model, a group has suggested the involvement of MMP-12 in the development of chronic asthma (FASEB, 2002, 16, A590). These results clearly demonstrate that human macrophage elastase (MMP-12) inhibitors might be very useful for preventing and treating chronic respiratory pathologies such as chronic obstructive pulmonary bronchitis (COPD), emphysema, chronic bronchitis and chronic pulmonary inflammation, and also respiratory pathologies caused by an inflammation phenomenon, such as asthma, mucoviscidosis, acute respiratory distress syndrome (ARDS), repiratory allergies including allergic rhinitis and also diseases associated with the production of TNFα including severe fibrotic pulmonary disease, pulmonary sarcoidosis and silicosis.

All metalloproteases have a catalytic domain consisting of 162 to 173 amino acids containing the active site of the enzyme. A Zn²⁺ ion is present in the active site, to which it is bound via histidine residues. This site is one of the preferred points of attachment of synthetic MMP inhibitors, since it especially allows the creation of a stable, powerful chelation centre that is readily accessible to small molecules. Thus, all the powerful inhibitors described in the literature contain a chemical function such as a hydroxamic acid allowing chelation between the zinc atom of the catalytic site of the metalloprotease and the said inhibitor. This chelation ensures blockage of the active site and results in inhibition of the said enzyme.

One of the major problems of inhibition of this type is the absence of selectivity or the low degree of selectivity, since all MMPs contain a zinc ion in their active site. The second problem associated with these powerful but generally poorly selective inhibitors is the toxicity associated with the presence of a chemical function such as a hydroxamic acid.

One of the objects of the invention is thus to provide novel compounds that have inhibitory properties on type 12 metalloprotease (MMP-12). A solution has been found by producing novel thiophene amino acid derivatives, and also by using the said compounds in pharmaceutical compositions that can be used in the prevention and treatment of pathologies associated with an inhibition of MMP-12.

Several scientific articles and patent applications describe compounds comprising a central thiophene unit. Among this literature, mention may be made of patent application WO 98/23605, which describes thien-2-ylcarboxamide derivatives substituted in position 4 with a cyclic system and in position 5 with a trifluoromethyl group. These compounds are claimed for their bactericidal and fungicidal activity. Patent application WO 96/16954 also describes compounds optionally comprising a 4-aryl-thien-2-ylcarboxamide system in which the amide function may be substituted with a phenyl group, which are useful for their antifungal properties.

None of these documents describes or suggests for these compounds inhibitory activity on MMP-12 or mixed MMP-12-13 inhibitory activity and a potential use of this type of product in the treatment of respiratory pathologies, or of pathologies of inflammatory type, which is a novel property of the compounds claimed by the applicant.

### SUMMARY OF THE INVENTION

The invention provides compounds of Formula (I), 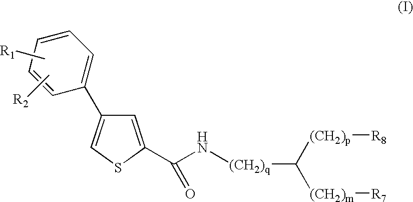
stereoisomers thereof, or pharmaceutically acceptable salts of said compounds or stereoisomers, wherein R₁, R₂, m, p, q, R₇ and R₈ are as defined below, as well as compositions comprising the same, processes for making the same, and methods of using the same to treat a variety of diseases, including, those requiring interaction with metalloproteases, and more specifically with macrophage metalloelastase (MMP-12), and for the prevention and treatment of respiratory pathologies such as chronic obstructive bronchopneumopathy (COPD), emphysema, chronic bronchitis, chronic pulmonary inflammation, asthma, cystic fibrosis, acute respiratory distress syndrome (ARDS), respiratory allergies including allergic rhinitis, and also diseases associated with the production of TNFα including severe fibrotic pulmonary disease, pulmonary sarcoidosis and silicosis. The compounds of the present invention also show inhibitory activity on metalloprotease-13 (MMP-13), making them useful for the treatment of pathologies involving this enzyme, such as cancer, osteoporosis, osteoarthritis, arthritis, rheumatoid arthritis, atherosclerosis, multiple sclerosis and cardiac insufficiency.

### DETAILED DESCRIPTION

The invention provides compounds of Formula (I), 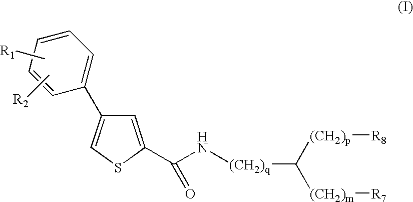
in which:
R₁ and R₂, which may be identical or different, independently of each other, each represent a group selected from:
hydrogen, halogen, cyano, nitro, halo(C₁-C₆)alkyl, halo(C₁-C₆)alkoxy, (C₁-C₆)alkyl, (C₂-C₆)alkenyl, (C₂-C₆)alkynyl, —OR₄, —NR₄R₅, —S(O)ₙR₄, —C(O)R₄, —CO₂R₄, —O—C(O)R₄, —C(O)NR₄R₅, —NR₅—C(O)R₄, —NR₅—SO₂R₄, -T-CN, -T-OR₄, -T-OCF₃, -T-NR₄R₅, -T-S(O)ₙR₄, -T-C(O)R₄, -T-CO₂R₄, -T-O—C(O)R₄, -T-C(O)NR₄R₅, -T-NR₅—C(O)R₄, -T-NR₅—SO₂R₄, —R₆, and -T-R₆ in which:
n represents an integer between 0 and 2 inclusive,
T represents a linear or branched (C₁-C₆)alkylene chain optionally substituted with one group selected from oxo, halogen, (C₁-C₆)alkoxy, hydroxyl, amino, mono(C₁-C₆)alkylamino and di(C₁-C₆)alkylamino, and/or in which one of the carbon atoms is optionally replaced with an oxygen atom, a sulphur atom, an —NH— group or an —N(C₁-C₆)alkyl- group (it being understood that when one of the carbon atoms is replaced with a group as defined above, then the said alkylene chain comprises at least a sequence of two atoms)
R₄ represents a hydrogen atom, a (C₁-C₆)alkyl, aryl group, cycloalkyl group, or a heterocycle,
R₅ represents a hydrogen atom or a (C₁-C₆)alkyl group,
R₆ represents a group selected from aryl, cycloalkyl and a heterocyle, each of these groups optionally being substituted with one to five groups, which may be identical or different, selected independently of each other from halogen, cyano, nitro, oxo, halo(C₁-C₆)alkyl, halo(C₁-C₆)alkoxy, (C₁-C₆)alkyl, (C₁-C₆)alkenyl, —OR₄₀, —NR₄₀R₅₀, —S(O)ₙ₁R₄₀, —C(O)R₄₀, —CO₂R₄₀, —O—C(O)R₄₀, —C(O)NR₄₀R₅₀, —NR₅₀—C(O)R₄₀, —NR₅₀—SO₂R₄₀, -T₁—CN, -T₁-OR₄₀, -T₁-OCF₃, -T₁-NR₄₀R₅₀, -T₁-S(O)ₙR₄₀-T₁-C(O)R₄₀, -T₁-CO₂R₄₀, -T₁-O—C(O)R₄₀, -T₁-C(O)NR₄₀OR₅₀, -T₁-NR₅₀—C(O)R₄₀ and -T₁-NR₅₀—SO₂R₄₀ in which R₄₀, R₅₀, T, and n₁ have the same meanings as R₄, R₅, T and n, respectively, as defined above;
m represents an integer between 0 and 4 inclusive;
p represents an integer between 0 and 4 inclusive;
q represents an integer between 0 and 4 inclusive;
R₇ represents a group selected from aryl, cycloalkyl and a heterocycle, each of these groups being optionally substituted with one to three groups, which may be identical or different, selected independently of each other from halogen, cyano, nitro, halo(C₁-C₆)alkyl, halo(C₁-C₆)alkoxy, (C₁-C₆)alkyl, hydroxyl, (C₁-C₆)alkoxy, amino, mono(C₁-C₆)alkylamino, di(C₁-C₆)alkylamino, mercapto, (C₁-C₆)alkylthio, (C₁-C₇)acyl, (C₁-C₆)alkyl sulphoxide, carboxyl and (C₁-C₆)alkoxycarbonyl;
R₈ represents a group selected from carboxyl, (C₁-C₆)alkoxycarbonyl, (C₁-C₇)acyl, hydroxy(C₁-C₆)alkylcarbonyl, (C₁-C₆)alkoxy(C₁-C₆)alkylcarbonyl, tetrazole, amino, aminocarbonyl and amino(C₁-C₆)alkylcarbonyl (the amino portion in each of the groups bearing this function being optionally substituted with one or two groups, which may be identical or different, selected independently of each other from (C₁-C₆)alkyl and cycloalkyl);
the isomers thereof and the addition salts thereof with a pharmaceutically acceptable acid or base, it being understood that in the general definition of the various groups in the compounds of formula (I):
the term “aryl” means an aromatic monocyclic or bicyclic system containing from 4 to 10 carbon atoms, it being understood that in the case of a bicyclic system, one of the rings is of aromatic nature and the other ring is aromatic or unsaturated; as a guide, mention may be made of the following groups: phenyl, naphthyl, indenyl, benzocyclobutenyl, 1,2,3,4-tetrahydronaphthyl, etc.;
the term “cycloalkyl” means a saturated or partially unsaturated, fused or bridged monocyclic or bicyclic system containing from 3 to 12 carbon atoms; as a guide, mention may be made of the following groups: cyclopropyl, cyclobutyl, cyclopentyl, cyclohexyl, cycloheptyl, cyclooctyl, adamantyl, decalinyl, norbornyl, cyclopentenyl, cyclohexenyl, cyclohexenediyl, etc.;
the term “heterocycle” means a saturated, unsaturated or aromatic, 3- to 12-membered fused or bridged monocyclic or bicyclic system comprising from 1 to 4 hetero atoms, which may be identical or different, selected independently of each other from oxygen, sulphur and nitrogen, and optionally containing 1 or 2 oxo or thioxo groups, it being understood that in the case of a bicyclic system, one of the rings may be of aromatic nature and the other ring is aromatic or unsaturated, or both rings are saturated, or one of the rings is saturated and the other ring is unsaturated, or both rings are unsaturated; as a guide, mention may be made of the following groups: furyl, thienyl, pyrrolyl, pyrazolyl, pyridyl, pyrimidyl, pyrazinyl, benzofuryl, benzothienyl, indolyl, quinolyl, isoquinolyl, imidazolyl, benzodioxolyl, benzodioxinyl, benzo[1,2,5]thiadiazolyl, benzo[1,2,5]oxadiazolyl, [1,2,3]triazolyl, [1,2,4]triazolyl, morpholinyl, piperidyl, piperazinyl, pyrrolidinyl, etc.;
the term “(C₁-C₆)alkyl” means a linear or branched carbon-based chain containing from 1 to 6 carbon atoms; as a guide, mention may be made of the following groups: methyl, ethyl, propyl, isopropyl, tert-butyl, neopentyl, hexyl, etc.;
the term “(C₂-C₆)alkenyl” means a linear or branched carbon-based chain containing from 2 to 6 carbon atoms and one or more double bonds; as a guide, mention may be made of the following groups: vinyl, allyl, 3-buten-1-yl, 2-methyl-buten-1-yl, hexenyl, etc.;
the term “(C₂-C₆)alkyenyl” means a linear or branched carbon-based chain containing from 2 to 6 carbon atoms and one or more triple bonds; as a guide, mention may be made of the following groups: ethynyl, propynyl, 3-butyn-1-yl, 2-methylbutyn-1-yl, hexynyl, etc.;
the term “(C₁-C₆)alkoxy” means an alkyl group as defined above linked via an oxygen atom; as a guide, mention may be made of the following groups: methoxy, ethoxy, n-propyloxy, tert-butyloxy, etc.;
the term “halo(C₁-C₆)alkyl” means a linear or branched carbon-based chain containing from 1 to 6 carbon atoms and substituted with 1 to 6 halogen atoms; as a guide, mention may be made of the following groups: trifluoromethyl, 2,2,2-trifluoroethyl, etc.;
the term “halo(C₁-C₆)alkoxy” means a linear or branched carbon-based chain containing from 1 to 6 carbon atoms and substituted with 1 to 6 halogen atoms, the said chain being linked to the compound of formula (I) via an oxygen atom; as a guide, mention may be made of the following groups: trifluoromethoxy, 2,2,2-trifluoroethoxy, etc.;
the term “halogen atom” means an atom selected from bromine, chlorine, fluorine and iodine;
the term “acyl” means a hydrogen atom, an alkyl group as defined above, a cycloalkyl of 3 to 6 carbon atoms or a phenyl group linked via an oxo group to the compounds of formula (I); as a guide, mention may be made of the following groups: formyl, acetyl, ethylcarbonyl, n-propylcarbonyl, tert-butylcarbonyl, cyclopropylcarbonyl, benzoyl, etc.;
the term “cyclic system” means aryl group, cycloalkyl group and heterocycles as defined above;
the optical isomers refer to the racemic mixtures, enantiomers and diastereoisomers.

According to one advantageous variant of the invention, the preferred compounds of the invention are the compounds of formula (I) in which:
R₁ represents a group selected from halo(C₁-C₆)alkoxy, (C₁-C₆)alkyl, —OR₄, —SR₄ and —R₆ in which:
R₄ represents a hydrogen atom or a (C₁-C₆)alkyl group,
R₆ represents a group selected from phenyl, cyclohexyl and a heterocycle, each of these groups being optionally substituted with one or two groups, which may be identical or different, selected independently of each other from halogen, halo(C₁-C₆)alkyl-, halo(C₁-C₆)alkoxy-, (C₁-C₆)alkyl, cyano, -T₁-CN, —OR₄₀, -T₁-OR₄₀, —NR₄₀R₅₀, —S(O)ₙ₁R₄₀ and —C(O)R₄₀, in which:
R₄₀ represents a group selected from a hydrogen atom, (C₁-C₆)alkyl and phenyl,
R₅₀ represents a group selected from a hydrogen atom and (C₁-C₆)alkyl,
T₁ represents a linear or branched (C₁-C₆)alkylene chain,
n₁ represents an integer between 0 and 2 inclusive;
R₂ represents a hydrogen atom;
m represents an integer between 0 and 4 inclusive;
p represents an integer between 0 and 4 inclusive;
q represents an integer between 0 and 4 inclusive;
R₇ represents a group selected from phenyl, monocyclic cycloalkyl and a 5- or 6-membered monocyclic heterocycle comprising one or two hetero atoms, which may be identical or different, selected independently of each other from oxygen and nitrogen, each of these groups being optionally substituted with one or two groups, which may be identical or different, selected independently of each other from halogen, cyano, nitro, halo(C₁-C₆)alkyl, halo(C₁-C₆)alkoxy, (C₁-C₆)alkyl, hydroxy, (C₁-C₆)alkoxy, amino, mono(C₁-C₆)alkylamino, di(C₁-C₆)alkylamino, mercapto, (C₁-C₆)alkylthio, (C₁-C₇)acyl, (C₁-C₆)alkyl sulphoxide, carboxyl and (C₁-C₆)alkoxycarbonyl;
R₈ represents a group selected from carboxyl, (C₁-C₆)alkoxycarbonyl, aminocarbonyl, (C₁-C₆)alkylcarbonyl, amino, hydroxy(C₁-C₆)alkylcarbonyl, (C₁-C₆)alkoxy(C₁-C₆)alkylcarbonyl and amino(C₁-C₆)alkylcarbonyl, (the amino portion in each of the groups bearing this function being optionally substituted with one or two (C₁-C₆)alkyl groups, which may be identical or different, independently of each other).

According to one particularly advantageous variant of the invention, the phenyl group in the compounds of formula (1) is substituted with a group R₁ as defined in the formula (1), located in the para position.

The groups R₁ that are preferred according to the invention are groups selected from trifluoromethoxy, 4-acetylphenyl, 4-pyridyl, 3-pyridyl, N-pyrrolidinyl, 1-methylpyrrol-3-yl, 3,6-dihydro-2H-pyrid-1-yl, cyclohexyl, 2-hydroxy-4-pyridyl and 2-hydroxyphenyl.

Very advantageously, R₁ represents a group selected from trifluoromethoxy, 4-acetylphenyl, cyclohexyl and 4-pyridyl.

Preferably, q is an integer equal to zero.

According to one advantageous variant of the invention, m is an integer selected from zero and one.

Similarly, for the preferred compounds of the invention, p is advantageously an integer selected from zero and one.

The group R₇ that is preferred according to the invention is the phenyl group.

The groups R₈ that are preferred according to the invention are groups selected from carboxyl and aminocarbonyl.

The isomers, and also the addition salts with a pharmaceutically acceptable acid or base, of the variants and the preferred compounds form an integral part of the invention.

The invention also relates to the pharmaceutically acceptable salts of the compounds of formula (I). A review of pharmaceutically acceptable salts is described especially in J. Pharm. Sci., 1977, 66, 1-19.

The expression “pharmaceutically acceptable acids” means non-toxic organic or mineral acids. Among the pharmaceutically acceptable acids that may be mentioned, without any limitation, are hydrochloric acid, hydrobromic acid, sulphuric acid, phosphonic acid, nitric acid, acetic acid, trifluoroacetic acid, lactic acid, pyruvic acid, malonic acid, succinic acid, glutaric acid, fumaric acid, tartaric acid, maleic acid, citric acid, ascorbic acid, oxalic acid, methanesulphonic acid, camphoric acid, benzoic acid, toluenesulphonic acid, etc.

The expression “pharmaceutically acceptable bases” means non-toxic organic or mineral bases.

Among the pharmaceutically acceptable bases that may be mentioned, without any limitation, are sodium hydroxide, potassium hydroxide, lithium hydroxide, calcium hydroxide, triethylamine, tert-butylamine, 2-diethylaminoethanol, ethanolamine, ethylenediaamine, dibenzylethylenediamine, piperidine, pyrrolidine, morpholine, piperazine, benzylamine, arginine, lysine, histidine, glucamine, glucosamine, quaternary ammonium hydroxides, etc.

In general, the expression “isomers of the compounds of the invention” means optical isomers such as enantiomers and diastereoisomers. More particularly, the pure enantiomeric forms of the compounds of the invention may be separated from mixtures of enantiomers that are reacted with a releasable agent for resolving the racemic mixtures, the said agent itself existing in the form of a pure enantiomer, allowing the corresponding diastereoisomers to be obtained. These diastereoisomers are then separated according to the separation techniques that are well known to those skilled in the art, such as crystallization or chromatography, and the resolving agent is then removed using the standard techniques of organic chemistry, to produce a pure enantiomer. In another manner, the pure enantiomeric forms of the compounds of the invention may be separated by chromatography on a chiral column.

The compounds of the invention that are present in the form of a mixture of diastereoisomers are isolated in pure form by using standard separation techniques such as chromatographies.

In certain particular cases, the process for separating the compounds of the invention may lead to the predominant formation of one enantiomer or one diastereoisomer relative to the other.

The invention also covers the process for preparing the compounds of formula (I). More particularly, the compounds of formula (I) may be obtained from the compounds of formula (II): 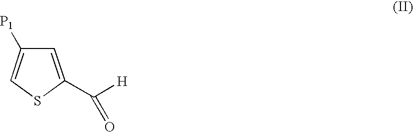
in which P₁ represents a halogen atom or a triflate group,
which compounds of formula (II) are subjected to oxidation conditions in the presence, for example, of silver nitrate in a basic and polar medium, to give the compounds of formula (III): 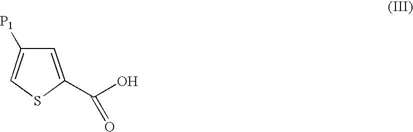
in which P₁ is as defined above,
which compounds of formula (III) are optionally converted into the corresponding acid chlorides (IV) by the action of oxalyl chloride, for example, 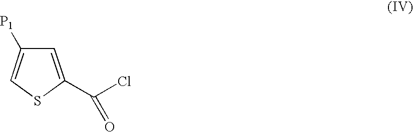
in which P₁ is as defined above,
or are treated directly, under peptide coupling conditions in the presence of a coupling agent and in a basic medium, with a compound of formula (V): 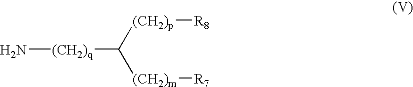
in which R₇, R₈, m, p and q have the same meanings as in the compounds of formula (I),
to give the compounds of formula (VI): 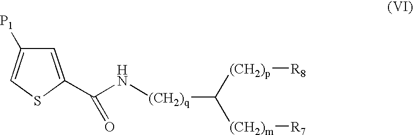
in which P₁, R₇, R₈, m, p and q are as defined above,
which compounds of formula (VI) are:
either reacted, under basic palladium coupling conditions, with a compound of formula (VII): 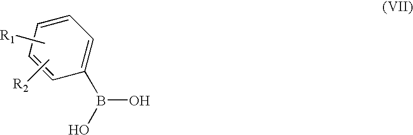
in which R₁ and R₂ have the same meanings as in the compounds of formula (I), to give the compounds of formula (I);
or treated with hexamethylditin, in the presence of a palladium catalyst, to give the compounds of formula (VII): 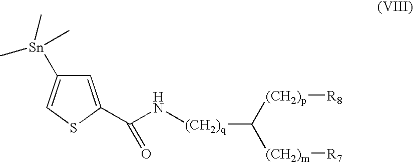
in which R₇, R₈, m, p and q are as defined above,
   which compounds of formula (VIII) are reacted:
with a compound of formula (IX): 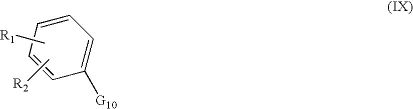
in which R₁ and R₂ have the same meanings as in formula (I) and G₁₀ represents a halogen atom selected from chlorine and bromine or a triflate group,
either in the presence of triphenylphosphinearsenic and a palladium catalyst, in the case where G₁₀ represents a triflate group,
or in the presence of a halocupric compound such as CuBr₂ and a palladium catalyst, under polar solvent conditions, in the case where G₁₀ represents a halogen atom,
also to give the compounds of formula (I);
or treated with bis(pinacolato)diborane followed by an oxidation reaction to give the compounds of formula (VIa): 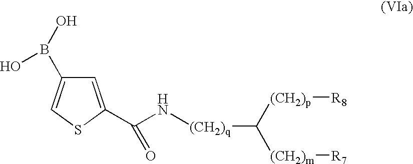
in which P₁, R₇, R₈, m, p and q are as defined above,
which compounds of formula (VIa) are reacted under basic palladium coupling conditions with a compound of formula (IX): 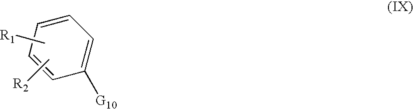
in which R₁, R₂ and G₁₀ are as defined above,
also to give the compounds of formula (I);
or reacted with a compound of formula (IXa): 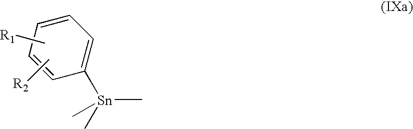
in which R₁, and R₂ are as defined in formula (I),
either in the presence of triphenylphosphinearsenic and a palladium catalyst, in the case where P₁ in the compounds of formula (VI) represents a triflate group,
or in the presence of a halocupric compound such as CuBr₂ and a palladium catalyst, under polar solvent conditions, in the case where P₁ in the compounds of formula (VI) represents a halogen atom,
also to give the compounds of formula (I;
compounds of formula (I) in the particular case where they represent compounds of formula (I/a) in which R₂ represents a hydrogen atom, R₁ represents a hydroxyl group or a halogen atom (Hal) and R₇, R₈, m, p and q have the same meanings as in formula (I): 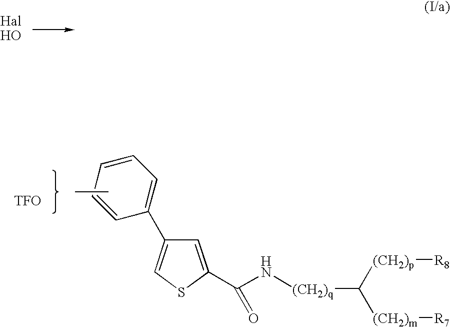
which compounds of formula (1/a) may then be pretreated with trifluoromethanesulphonic anhydride in the presence of a strong base, in the case where R₁ represents a hydroxyl group, to obtain the activated triflate derivative, it being possible for the said compounds bearing a group R₁ taking the halogen or triflate definition to be then
either reacted under basic conditions and in the presence of a palladium catalyst with a compound of formula (X): 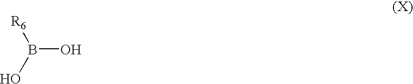
in which R₆ is as defined in formula (I), that means it represents a group selected from aryl, cycloalkyl and a heterocycle,
to give the compounds of formula (I/b), a particular case of the compounds of formula (I): 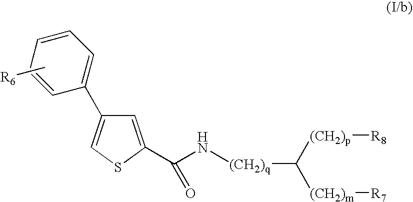
in which R₆, R₇, R₈, m, p and q are as defined above,
or treated with bis(pinacolato)diborane followed by an oxidation reaction, to give the compounds of formula (XI): 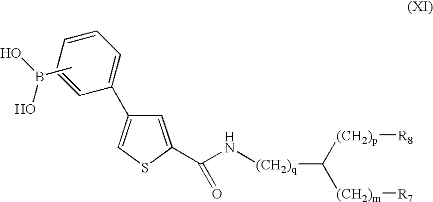
in which R₇, R₈, m, p and q are as defined above, which compounds of formula (XI) are reacted under basic conditions and in the presence of a palladium catalyst with a compound of formula (Xa):
   R₆—P₂ (Xa)
in which R₆ is as defined in formula (I), that means it represents a group selected from aryl, cycloalkyl and a heterocycle, and P₂ represents a halogen atom or a triflate group,
also to give the compounds of formula (I/b);
or treated with hexamethylditin, in the presence of a palladium catalyst, to give the compounds of formula (XIa): 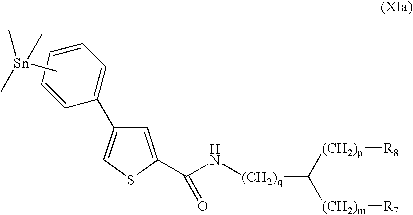
in which R₇, R₈, m, p and q are as defined above, which compounds of formula (XIa) are reacted with a compound of formula (Xa) as defined above:
   R₆—P₂ (Xa)
in which R₆ is as defined in formula (I) and P₂ represents a halogen atom or a triflate group,
either in the presence of triphenylphosphinearsenic and a palladium catalyst, in the case where P₂ represents a triflate group,
or in the presence of a halocupric compound such as CuBr₂ and a palladium catalyst, under polar solvent conditions, in the case where P₂ represents a halogen atom,
also to give the compounds of formula (I/b);
or reacted with a compound of formula (Xb):
   R₆—SnMe₃ (Xb)
in which R₆ is as defined above,
either in the presence of triphenylphosphinearsenic and a palladium catalyst, in the case where the compounds of formula (I/a) comprise a triflate group,
or in the presence of a halocupric compound such as CuBr₂ and a palladium catalyst, under polar solvent conditions, in the case where the compounds of formula (I/a) comprise a halogen atom,
also to give the compounds of formula (I/b);
or reacted under palladium coupling conditions, in basic medium, with a compound of formula (XII):
   R_{6′}—H (XII)
in which R_{6′} represents a nitrogenous heterocycle optionally substituted with one or more groups as defined for the substituents of the group R₆ in the compounds of formula (I),
to give the compounds of formula (I/c), a particular case of the compounds of formula (I): 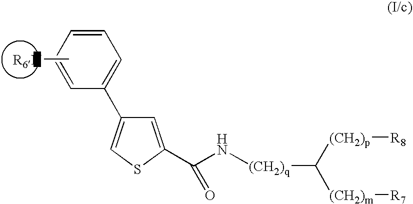
in which R₇, R₈, m, p and q are as defined above, and R_{6′} represents an optionally substituted nitrogenous heterocycle as defined in formula (I),
the compounds (I/a) to (I/c) together forming compounds of the invention, which are purified, where appropriate, according to a standard purification technique, which may be, if so desired, separated into the various isomers thereof according to a standard separation technique, and which are converted, where appropriate, into the addition salts thereof with a pharmaceutically acceptable acid or base.

The compounds of formulae (II), (V), (VII), (IX), (X) and (Xa) are either commercial compounds or are obtained according to known methods of organic synthesis that are readily available and comprehensibles to those skilled in the art.

According to one variant of the invention, the compounds of formula (1) may also be obtained via a second preparation process characterized in that the starting material used is a compound of formula (II): 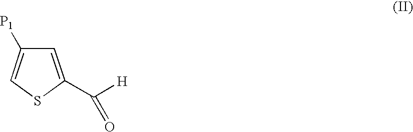
in which P₁ represents a halogen atom or a triflate group,
which compounds of formula (II) are subjected to oxidation conditions in the presence, for example, of silver nitrate in a basic and polar medium, to give the compounds of the formula (III): 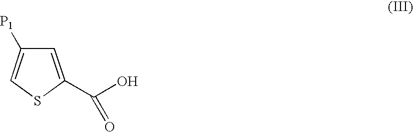
in which P₁ is as defined above,
in which compounds of formula (III) the acid function is esterified by the action of an alcohol in the presence of a strong acid, to give the compounds of formula (XX): 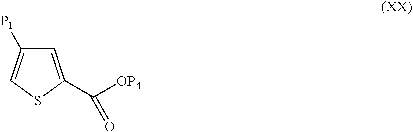
in which P₁ is as defined above, and P₄ represents a linear or branched (C₁-C₄)alkyl group,
which compounds of formula (XX) are:
either reacted, under basic palladium coupling conditions, with a compound of formula (VII): 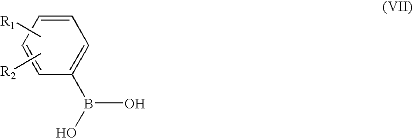
in which R₁ and R₂ have the same meanings as in the compounds of formula (1), to give the compounds of formula (XXa): 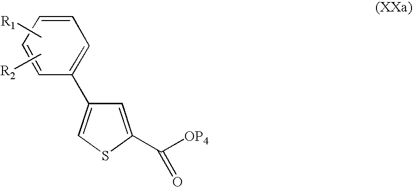
   in which R₁, R₂ and P₄ are as defined above,
or treated with hexamethylditin, in the presence of a palladium catalyst, to give the compounds of formula (XXI): 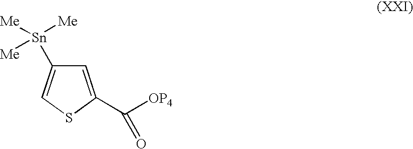
in which P₄ is as defined above,
which compounds of formula (XXI) are reacted:
with a compound of formula (IX): 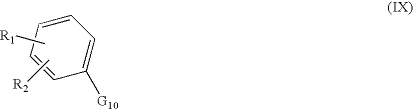
in which R₁ and R₂ have the same meanings as in formula (I) and G₁₀ represents a halogen atom selected from chlorine and bromine or a triflate group,
either in the presence of triphenylphosphinearsenic and a palladium catalyst, in the case where G₁₀ represents a triflate group,
or in the presence of a halocupric compound such as CuBr₂ and a palladium catalyst, under polar solvent conditions, in the case where G₁₀ represents a halogen atom,
also to give the compounds of formula (XXa) as described above;
or treated with bis(pinacolato)diborane, followed by an oxidation reaction, to give the compounds of formula (XXII): 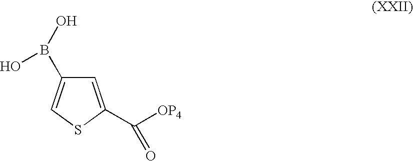
   in which P₄ is as defined above,
which compounds of formula (XXII) are reacted under basic palladium coupling conditions with a compound of formula (IX) as described above,
also to give the compounds of formula (XXa) as defined above;
or reacted with a compound of formula (IXa) (the said compounds of formula (Ixa) being obtained by treating the compounds of formula (IX) as defined above, with hexamethylditin in the presence of a palladium catalyst): 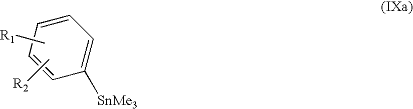
in which R₁, and R₂ are as defined in formula (I),
either in the presence of triphenylphosphinearsenic and a palladium catalyst, in the case where P₁ in the compounds of formula (XX) represents a triflate group,
or in the presence of a halocupric compound such as CuBr₂ and a palladium catalyst, under polar solvent conditions, in the case where P₁ in the compounds of formula (XX) represents a halogen atom,
also to give the compounds of formula (XXa) as defined above;
which compounds of formula (XXa) are saponified under basic hydrolysis conditions,
to give the compounds of formula (XXb): 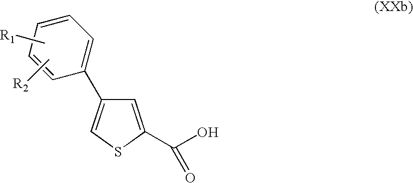
in which R₁ and R₂ are as defined in formula (I),
which compounds of formula (XXb) are either converted beforehand into the corresponding acid chloride by the action of oxaylyl chloride, or treated directly, under peptide coupling conditions in the presence, for example, of a coupling agent and in a basic medium, with a compound of formula (V): 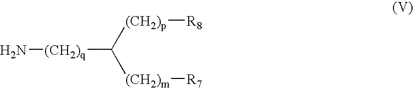
in which R₇, R₈, m, p and q have the same meanings as in the compounds of formula (I),
to give the compounds of formula (1): 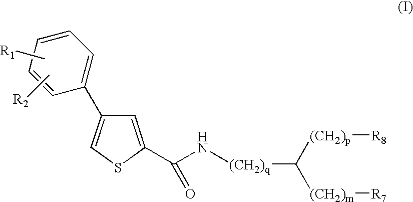
compounds of formula (I), which are purified, where appropriate, according to a standard purification technique, which may be, if so desired, separated into the various isomers thereof according to a standard separation technique, and which are converted, where appropriate, into the addition salts thereof with a pharmaceutically acceptable acid or base.

On account of their pharmacological properties as MMP-12 inhibitors, the compounds of the present invention are useful in the prevention and treatment of respiratory pathologies such as chronic obstructive bronchopneumopathy (COPD), emphysema, chronic bronchitis, chronic pulmonary inflammation, asthma, mucoviscidosis, acute respiratory distress syndrome (ARDS), respiratory allergies including allergic rhinitis, and also diseases associated with the production of TNFα, including severe fibrotic pulmonary disease, pulmonary sarcoidosis and silicosis. The compounds of the present invention also show, to a lesser extent, inhibitory activity on metalloprotease-13 (MMP-13), making them potentially useful for the treatment of pathologies involving this enzyme, such as cancer, osteoporosis, osteoarthritis, arthritis, rheumatoid arthritis, atherosclerosis, multiple sclerosis and cardiac insufficiency.

Advantageously, the compounds of the present invention are useful for preventing and treating chronic obstructive bronchopneumopathy, emphysema and chronic bronchitis.

More particularly, the compounds of the present invention are useful for treating tobacco-related emphysema.

According to one variant of the invention, the compounds of formula (I) are useful for preventing and treating asthma.

The subject of the present invention is also pharmaceutical compositions containing as active principle at least one compound of formula (I), an isomer thereof or an addition salt thereof with a pharmaceutically acceptable acid or base, alone or in combination with one or more inert, non-toxic, pharmaceutically acceptable excipients or vehicles.

Among the pharmaceutical compositions according to the invention, mention will be made more particularly of those that are suitable for oral, parenteral (intravenous, intramuscular or subcutaneous), percutaneous or transcutaneous, intravaginal, rectal, nasal, perlingual or respiratory administration.

The pharmaceutical compositions according to the invention for parenteral injections especially comprise aqueous and non-aqueous sterile solutions, dispersions, suspensions or emulsions and also sterile powders to reconstitute injectable solutions or dispersions.

The pharmaceutical compositions according to the invention for solid oral administration especially comprise simple or sugar-coated tablets, sublingual tablets, sachets, gel capsules and granules, and, for oral, nasal or buccal liquid administration, especially comprise emulsions, solutions, suspensions, drops, syrups and aerosols.

The pharmaceutical compositions according to the invention for administration via the respiratory route especially comprise compositions in the form of solutions for aerosols or powders for inhalers. When the compositions are aerosols, for the use of liquid aerosols, the compositions may be stable sterile solutions or solid compositions dissolved at the time of use in apyrogenic sterile water, in physiological saline or in any other pharmaceutically acceptable vehicle. For use in the form of dry aerosols intended to be inhaled directly, the active principle is optionally finely divided or micronized, and combined with an inert, solid, water-soluble diluent or vehicle.

The pharmaceutical compositions for rectal administration are preferably suppositories, and those for percutaneous or transcutaneous administration especially comprise powders, aerosols, creams, ointments, gels and patches.

The pharmaceutical compositions mentioned above illustrate the invention but do not limit it in any way.

Among the inert, non-toxic, pharmaceutically acceptable excipients or vehicles that may be mentioned, as a guide and with no limitation, are diluents, solvents, preserving agents, wetting agents, emulsifiers, dispersants, binders, swelling agents, crumbling agents, retardants, lubricants, absorbing agents, suspension agents, colorants, flavourings, etc.

The practical dosage varies according to the age and weight of the patient, the route of administration, the pharmaceutical composition used, the nature and severity of the complaint, and the possible taking of associated treatments. The dosage ranges from 1 mg to 1000 mg in one or more dosage intakes per day.

The examples that follow illustrate the invention but do not limit it in any way.

The starting materials used are commercial products or products prepared according to known procedures from commercial compounds or compounds known to those skilled in the art. The various preparations give synthetic intermediates that are useful for preparing the compounds of the invention.

The structures of the compounds described in the examples and in the preparations were determined according to the usual spectrophotometric techniques (infrared (1R), nuclear magnetic resonance (NMR), mass spectrometry (MS) including electron spray (ES) mass spectrometry, etc.) and the purity was determined by high performance liquid chromatography (HPLC).

Abbreviations used in the procedures:
TOTU: O-[(ethoxycarbonyl)cyanomethylamino]-N—N—N′-N′-tetramethyluronium fluoroborate;
DME: 1,2-dimethoxyethane (or ethylene glycol dimethyl ether)
TFA: trifluoroacetic acid
HATU: O-(7-azabenzotriazol-1-yl)-N,N,N′,N′-tetramethyluronium hexafluorophosphate
tBME: tert-butyl methyl ether
   Preparation 1: 4-[4-(Trifluoromethoxy)phenyl]thiophen-2-carboxylic acid
   Stage 1: 4-[4-(Trifluoromethoxy)phenyl]thiophene-2-carbaldehyde

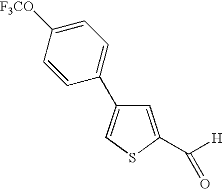

84.9 ml (2.1 equivalents) of a 2.0M solution of potassium phosphate and 2.8 g (0.03 equivalent) of tetrakis(triphenylphosphine)palladium(0) are added to a solution of 12.3 g of 4-bromothiophene-2-carbaldehyde and 20.0 g of [4-(trifluoromethoxy)phenyl]-boronic acid (1.2 equivalents) in 70 ml of degassed DME. The reaction medium is stirred for 3 hours at 80° C. and then concentrated under reduced pressure. The residue obtained is taken up in ethyl acetate. The solution is then filtered through Celite, washed with water, dried over sodium sulphate, filtered and then concentrated under reduced pressure. Chromatography of the residue on silica gel (9/1 cyclohexane/ethyl acetate) allows 15.05 g of the expected product to be isolated.

Yield: 68%

¹H NMR (CDCl₃) δ (Ppm): 10.0 (s, 1H), 8.0 (s, 1H), 7.80 (s, 1H), 7.55 (m, 2H), 7.25 (m, 2H)
Stage 2: 4-[4-(Trifluoromethoxy)phenyl]thiophene-2-carboxylic acid

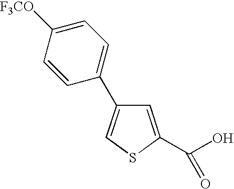

37.6 g (4 equivalents) of silver nitrate and 44.2 ml (8 equivalents) of aqueous 1.0M sodium hydroxide solution are added to a solution of 15.05 g of the compound obtained in stage 1 in 200 ml of ethanol. The reaction medium is stirred for 2 hours at 40° C., then filtered through Celite and concentrated under reduced pressure. The aqueous phase is washed with aqueous 1.0M hydrochloric acid solution, extracted with ethyl acetate, dried over sodium sulphate, filtered and concentrated under reduced pressure, to give 15.514 g of a beige-coloured powder corresponding to the expected product.

Yield: 97.4%

MS: MH⁻287
Preparation 2: (2S)-2-Amino-N-cyclohexyl-2-phenylacetamide

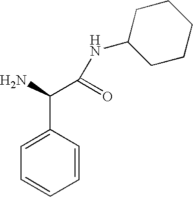

Stage 1: (2S)-tert-Butoxy-2-(cyclohexylamino)-2-oxo-1-phenylethyl carbamate

0.164 ml of cyclohexylamine (1.2 equivalents), 0.548g of O-[(ethoxycarbonyl)-cyanomethyleneamino]-N,N,N′,N′-tetramethyluronium (TOTU) and 830 μl of N-ethyl-N,N-diisopropylamine are added to a solution of 300 mg of (2S)-[(tert-butoxycarbonyl)amino](phenyl)acetic acid in 5 ml of anhydrous dimethylformamide. The reaction medium is stirred for 3 hours at room temperature and then concentrated under reduced pressure. The residue obtained is dissolved in ethyl acetate (30 ml), washed with water (30 ml), dried over sodium sulphate, filtered and concentrated under reduced pressure. Chromatography of the residue on silica gel (70/30: cyclohexane/ethyl acetate) allows 258 mg of the expected product to be isolated.

Yield: 65%

MS: MH⁺333

Stage 2: (2S)-2-Amino-N-cyclohexyl-2-phenylacetamide

0.297 ml of trifluoroacetic acid is added at 0° C. to a solution of 258 mg of the product obtained in stage 1 in 4 ml of anhydrous dichloromethane. The reaction medium is stirred for 17 hours at room temperature, washed with water (30 ml) and then with saturated sodium hydrogen carbonate solution (30 ml), dried over sodium sulphate, filtered and concentrated under reduced pressure. Recrystallization of the residue from diisopropyl ether allows 219 mg of the expected product to be isolated.

Yield: 99%

¹H NMR (CDCl₃) δ (Ppm): 7.30 (m, 5H), 6.90 (m, 1H), 4.50 (m, 1H), 4.10 (m, 2H), 3.80 (m, 1H), 2.50 (m, 1H), 1.60 (m, 12H)

MS: MH⁺233

HPLC: 97.1%
Preparation 3: Ethyl (3S)-3-amino-4-phenylbutanoate

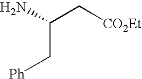

1.0 ml of concentrated sulphuric acid (10 volumes) is added to a solution of 0.5 g of (3S)-3-[(tert-butoxycarbonyl)amino]-4-phenylbutanoic acid in 10 ml of ethanol. The reaction medium is stirred for 17 hours at reflux. The solution is then concentrated under reduced pressure and basified with saturated sodium hydrogen carbonate solution to pH 8. The solution obtained is extracted with ethyl acetate and the organic phase is dried over sodium sulphate and then filtered to give 0.286 g of the expected product after evaporation under reduced pressure.

Yield: 77%

¹H NMR (DMSO) δ (ppm): 7.30 (m, 2H), 7.20 (m, 3H), 4.0 (q, 2H), 3.20 (m, 1H), 2.70 (m, 2H), 2.30 (m, 2H), 1.50 (s, 2H), 1.10 (t, 3H)

MS: MH⁺208

HPLC: 100%
Preparation 4: Methyl (4R)-4-amino-5-phenylpentanoate 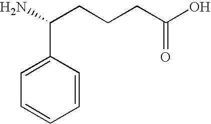
Stage 1: Methyl (3R)-3-[(tert-butoxycarbonyl)amino]-3-phenylpropanoate

1.9 ml of trimethylsilyldiazomethane as a 2.0M solution in cyclohexane are added to a solution of 0.31 g of (3R)-3-[(tert-butoxycarbonyl)amino]-3-phenylpropanoic acid, prepared according to the method described in Tetrahedron, 1997, 53, 12867-12874, in a mixture of 6 ml of ethyl ether and 6 ml of methanol. The reaction medium is stirred for 1 hour at room temperature and then hydrolysed with 100 μl of acetic acid and concentrated under reduced pressure. The crude product is triturated in a cyclohexane/ethyl acetate mixture (½) and filtered to give 0.386 g of the desired product.

Yield: 77%

NMR ¹H (DMSO) δ (ppm): 7.40 (m, 1H), 7.30 (m, 5H), 4.40 (m, 1H), 3.60 (s, 3H), 2.30 (m, 2H), 1.80 (m, 2H), 1.40 (s, 9H)

MS: MH⁺294

HPLC: 100%

Stage 2: Methyl (4R)-4-amino-5-phenylpentanoate

1.62 ml (10 equivalents) of a 2.1M solution of hydrochloric acid in methanol are added to a solution of 0.1 g of the product obtained in stage 1 in 2.0 ml of methanol at 0° C. The reaction medium is stirred for 1 hour at room temperature and concentrated under reduced pressure to give 73 mg of the desired product.

Yield: 93%

¹H NMR (DMSO) δ (ppm): 8.90 (m, 3H), 7.40 (m, 5H), 4.40 (m, 1H), 3.60 (s, 3H), 2.30 (m, 2H), 2.0 (m, 2H)

MS: MH⁺194
Preparation 5: (4-Cyclohexyl)phenylboronic acid 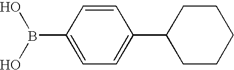
Stage 1: 4-[4-(Pinacolboro)phenyl]cyclohexyl

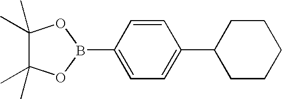

0.382 g of bis(pinacolato)diboron (1.2 equivalents), 27.5 mg of dichloro[[1,1′]-bis(diphenylphosphino)ferrocene]palladium(II) (0.03 equivalent), 41.7 mg of [1,1′]bis(diphenylphosphino)ferrocene (0.06 equivalent) and 0.369 g of potassium acetate (3 equivalents) are added, under nitrogen, to a solution of 0.3 g of bromophenylcyclohexyl in 5 ml of degassed 1,4-dioxane. The reaction medium is stirred for 17 hours at 80° C., diluted with ethyl acetate (100 ml), washed with water (3×60 ml), dried over sodium sulphate and concentrated under reduced pressure. Chromatography of the residue on silica gel (95/5 heptane/ethyl acetate) allows 0.2 g of the desired product to be obtained.

Yield: 56%

¹H NMR (DMSO) δ (ppm): 7.60 (d, 2H), 7.20 (d, 2H), 1.70 (m, 5H), 1.40 (m, 4H), 1.30 (m, 12H)

Stage 2: (4-Cyclohexyl)phenylboronic acid

3 ml of water and 0.23 g of sodium periodate (3.0 equivalents) are added to a solution of 0.103 g of the compound obtained in stage 1 above in 3 ml of acetone. The reaction medium is stirred for 17 hours at 60° C. and then concentrated under reduced pressure. The residue obtained is dissolved in ethyl acetate (20 ml), washed with 1.0M hydrochloric acid solution and then with water (3× ml), dried over sodium sulphate, filtered and then concentrated under reduced pressure. Chromatography of the residue on silica gel (90/10 dichloromethane/methanol) allows 64 mg of the desired product to be obtained.

Yield: 87%

MS: MH⁻(+HCO₂H) 249

HPLC: 100%
Preparation 6: Methyl 4-(4-bromophenyl)thiophene-2-carboxylate 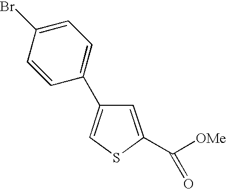
Stage 1: Methyl 4-(4-nitrophenyl)thiophene-2-carboxylate

The product (1.94 g) is obtained according to the process of stage 1 of preparation 1, using methyl 4-bromothiophene-2-carboxylate and (4-nitrophenyl)boronic acid as substrates.

Yield: 78%

¹H NMR (CDCl₃) δ (Ppm): 3.92 (s, 3H), 7.75 (d, 2H), 7.82 (s, 1H), 8.12 (s, 1H), 8.30 (d, 2H)

Stage 2: Methyl 4-(4-aminophenyl)thiophene-2-carboxylate

A solution of 1.94 g of the compound obtained in stage 1 above in 20 ml of methanol containing 194 mg of 10% palladium-on-charcoal is stirred in an autoclave for 6 hours at 50° C. under 10 bar of hydrogen. The reaction medium is then filtered through Celite and concentrated under reduced pressure to give 1.51 g of the desired product.

Yield: 88%

¹H NMR (DMSO) δ (ppm): 3.82 (s, 3H), 5.22 (s, 2H), 6.60 (d, 2H), 7.42 (d, 2H), 7.90 (s, 1H), 8.05 (s, 1H)

MS: MH⁺234

Stage 3: Methyl 4-(4-bromophenyl)thiophene-2-carboxylate

0.6 ml of concentrated hydrobromic acid is added to a solution of 103 mg of the product obtained in stage 2 above in 1.5 ml of water. The reaction medium is cooled to 0° C. and a solution of 35.5 mg of sodium nitrite (1.1 equivalents) in 0.5 ml of water is then added dropwise. After stirring for 1 hour at 0° C., a solution of 68 mg of copper bromide in 0.5 ml of concentrated hydrobromic acid is added dropwise. The reaction medium is stirred for a further 1 hour at 0° C. and then diluted with ethyl acetate (30 ml), washed with water (3×15 ml), washed with saturated sodium hydrogen carbonate solution (15 ml) and then washed again with water (15 ml). The organic phase is dried over sodium sulphate, filtered and then concentrated under reduced pressure. Chromatography of the residue on silica gel (95/5 cyclohexane/ethyl acetate) allows 52 mg of the desired product to be isolated.

Yield: 40%

¹H NMR (CDCl₃) δ (Ppm): 3.92 (s, 3H), 7.45 (d, 2H), 7.55 (d, 2H), 7.65 (s, 1H), 8.05 (s, 1H)

HPLC: 91.4%

### EXAMPLE 1

### Ethyl (2S){[4(4trifluoromethoxyphenyl)thien-2-yl]carboxamido}-(phenyl)acetate

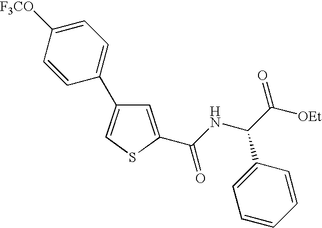

246 mg of ethyl (2S)-amino(phenyl)acetate hydrochloride (1.1 equivalents), 395 mg of O-(7-azabenzotriazol-1-yl)-N,N,N′,N′-tetramethyluronium hexafluorophosphate (HATU) and 362 μl of N-ethyl-N,N-diisopropylamine are added to a solution of 300 mg of the compound obtained in Preparation 1 in 6 ml of anhydrous dimethylformamide. The reaction medium is stirred for 17 hours at room temperature and then hydrolysed. The precipitate formed is filtered off, washed with water and finally dried overnight to give 639 mg of the expected product.

Yield: 100%

¹H NMR (DMSO) δ (ppm): 9.20 (d, 1H), 8.5 (s, 1H), 8.20 (s, 1H), 7.85 (d, 2H), 7.45 (m, 7H), 5.6 (d, 1H), 4.15 (m, 2H), 1.15 (t, 3H).

HPLC: 98.50%

### EXAMPLE 2

### (2S)({[4-(4Trifluoromethoxyphenyl)thien-2-yl]carboxamido}-(phenyl)acetic acid

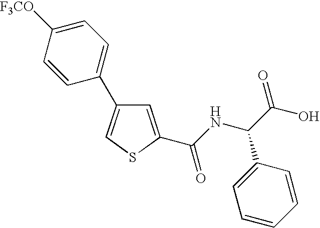

170 mg of lithium hydroxide (5 equivalents) and 200 R₁ of dimethylformamide are added to a solution of 639 mg of the compound obtained in Example 1 in 22 ml of an ethanol/water mixture (1/1). The reaction medium is stirred overnight at room temperature and then concentrated under reduced pressure. The solid obtained is taken up in water and acidified with 1.0M hydrochloric acid solution to pH 1. The precipitate formed is then filtered off, washed with water and then dried overnight to give 377 mg.

Yield: 64%

¹H NMR (DMSO) δ (ppm): 13.0 (bs, 1H), 8.94 (s, 1H), 8.49 (s, 1H), 8.17 (s, 1H), 7.84 (d, 2H), 7.43 (m, 7H), 5.44 (s, 1H)

MS: MH⁺422

HPLC: 98.4%

### EXAMPLE 3

### Ethyl (2R){[4-(4trifluoromethoxyphenyl)thien-2-yl]carboxamido}-(phenyl)acetate

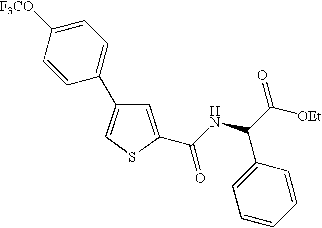

The product (200 mg) is obtained according to the process of Example 1, using ethyl (2R)-amino(phenyl)acetate hydrochloride as substrate.

Yield: 44%

MS: MH⁺436

HPLC: 98,96%

### EXAMPLE 4

### (2R)-{[4-(4-Trifluoromethoxyphenyl)thien-2-yl]carboxamido}-(phenyl)acetic acid

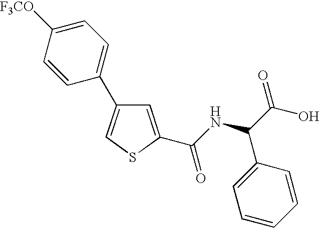

The product (121 mg) is obtained according to the process of Example 2, using the compound obtained in Example 3 as substrate.

Yield: 65%

¹H NMR (DMSO) δ (ppm): 13.01 (bs, 1H), 9.075 (d, 1H), 8.54 (s, 1H), 8.19 (s, 1H), 7.83 (d, 2H), 7.42 (m, 7H), 5.57 (d, 1H)

MS: MH⁺422

HPLC: 99.0%

### EXAMPLE 5

### Ethyl 3-{[4-(4-trifluoromethoxyphenyl)thien-2-yl]carboxamido)-3-phenylpropanoate

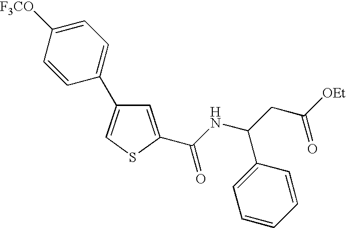

The product (283 mg) is obtained according to the process of Example 1, using ethyl amino(phenyl)propanoate hydrochloride as substrate.

Yield: 59%

MS: MH⁺464

HPLC: 96.69%

### EXAMPLE 6

### {1[(4-Trifluoromethoxyphenyl)thien-2-yl]carboxamido}(phenyl)-propanoic acid

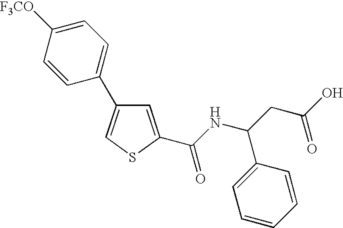

The product (198 mg) is obtained according to the process of Example 2, using the compound obtained in Example 5 as substrate.

Yield: 75%

¹H NMR (DMSO) δ (ppm): 9.03 (d, 1H), 8.29 (s, 1H), 8.15 (s, 1H), 7.82 (d, 2H), 7.46 (d, 2H), 7.41 (d, 2H), 7.34 (t, 2H), 7.25 (t, 1H), 5.40 (q, 1H), 2.83 (m, 2H)

MS: MH⁺436

HPLC: 99.3%

### EXAMPLES 7 and 8

### (2S){[4-(4-Trifluoromethoxyphenyl)thien-2-yl]carboxamido}(phenyl)propanoic acid and (2R){[4-(4-Trifluoromethoxyphenyl)thien-2-yl]carboxamido}(phenyl)propanoic acid

The products (60.9 mg and 50.2 mg) are obtained from the racemic mixture of Example 6 after a chiral separation on preparative HPLC under the following conditions:
Column: Chiralpack® AD-H 20×250 mm, 5 μm, temperature: ambiant, UV detection: 214 nm, flow rate: 20 mL/min,
Mobile phase: T=0, B=70% hexane, A=30% isopropanol containing 0.2% diethylamine (v/v); T=30 min, B=30%, A=70% (v/v).

### EXAMPLE 7

### (2S)-{[4-(4-Trifluoromethoxyphenyl)thien-2-yl]carboxamido}(phenyl)-propanoic acid

Yield: 31%

¹H NMR (DMSO) δ (ppm): 9.16 (bs, 1H), 8.29 (s; 1H), 8.14 (s, 1H), 7.82 (d, 2H), 7.45 (d, 2H), 7.40 (d, 2H), 7.33 (t, 2H), 7.24 (t, 1H), 5.38 (q, 1H), 2.79 (m, 2H)

MS: MH⁺436

HPLC: 99.21%

### EXAMPLE 8

### (2R)-{[4-(4-Trifluoromethoxyphenyl)thien-2-yl]carboxamido}(phenyl)-propanoic acid

Yield: 26%

¹H NMR (DMSO) δ (ppm): 12.51 (bs, 1H), 9.11 (m, 1H), 8.29 (s, 1H), 8.14 (s, 1H), 7.82 (d, 2H), 7.46 (d, 2H), 7.41 (d, 2H), 7.33 (t, 2H), 7.24 (t, 1H)

MS: MH⁺436

HPLC: 99.70%

### EXAMPLE 9

### N3-amino-3-oxo-1-phenylpropyl)-4-(4trifluoromethoxyphenyl) -thiophene-2-carboxamide

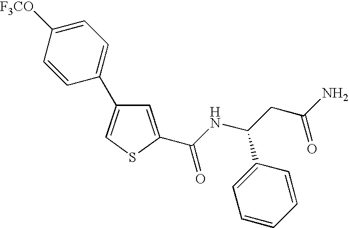

A large excess of 28% aqueous ammonia solution is added, at 0° C., to a solution of 335 mg of the compound obtained in Example 8 in 5 ml of anhydrous tetrahydrofuran. The ice bath is removed and the mixture is stirred overnight at room temperature. The crude reaction mixture is concentrated under reduced pressure, hydrolysed and extracted with dichloromethane. The organic phases are combined, dried over sodium sulphate and concentrated. Chromatography of the residue on silica gel (70/30: cyclohexane/ethyl acetate) allows 33 mg of the desired product to be obtained.

Yield: 15%

¹H NMR (DMSO) δ (ppm): 9.11 (d, 1H), 8.29 (s, 1H), 8.14 (s, 1H), 7.82 (d, 2H), 7.46 (m, 2H), 7.41 (m, 5H), 7.20 (m, 1H), 6.80 (m, 1H), 5.40 (m, 1H), 2.70 (m, 2H)

MS: MH⁻433

HPLC: 100%

### EXAMPLE 10

### 4-(4-Trifluoromethoxyphenyl)-N-[(2S)-2-(cyclohexylamino)-2-phenylethanoyl]thiophene-2-carboxamide

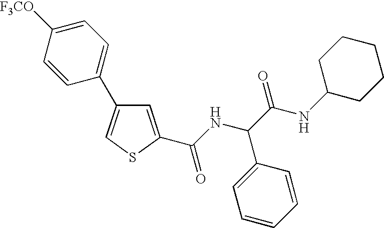

The product (265 mg) is obtained according to the process of stage 1 of Preparation 2, using the compounds obtained in Preparations 1 and 2 as substrates.

Yield: 56%

¹H NMR (DMSO) δ (ppm): 9.11 (m, 1H), 8.70 (s, 1H), 8.30 (m, 1H), 8.20 (s, 1H) 7.82 (d, 2H), 7.46 (m, 5H), 7.30 (m, 2H), 5.70 (d, 2H), 3.40 (m, 1H), 1.80 (m, 1H), 1.70 (m, 3H), 1.5 (m, 1H), 1.20 (m, 5H)

MS: MH⁺503

HPLC: 100%

### EXAMPLE 11

### Ethyl (3S)-3-{[4-(4-trifluoromethoxyphenyl)thien-2-yl]carboxamido}4-phenylbutanoate

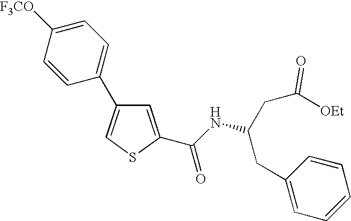

The product (191 mg) is obtained according to the process of Example 1, using the product of Preparation 3 as substrate.

Yield: 29%

¹H NMR (DMSO) δ (ppm): 8.70 (m, 1H), 8.20 (s, 1H), 8.10 (s, 1H), 7.82 (d, 2H), 7.46 (m, 2H), 7.30 (m, 5H), 4.70 (m, 1H), 4.20 (q, 2H), 2.90 (m, 2H), 2.60 (m, 2H), 1.0 (t, 3H)

MS: MH⁺478

HPLC: 100%

### EXAMPLE 12

### (3S)-3-{[4-(4-Trifluoromethoxyphenyl)thien-2-yl]carboxamido)-4-phenylbutanoic acid

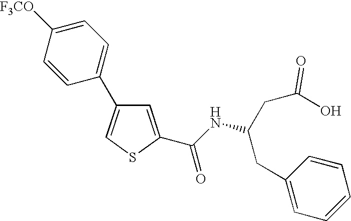

The product (104 mg) is obtained according to the process of Example 2, using the compound obtained in Example 11 as substrate.

Yield: 50%

¹H NMR (DMSO) δ (ppm): 8.90 (m, 1H), 8.20 (m, 2H), 7.82 (d, 2H), 7.46 (m, 2H), 7.30 (m, 5H), 4.40 (m, 1H), 2.90 (m, 2H), 2.60 (m, 2H)

MS: MH⁻448

HPLC: 100%

### EXAMPLE 13

### Methyl (4R)-3-{[4-(4-trifluoromethoxyphenyl)thien-2-yl]carboxamido}-4-phenylpentanoate

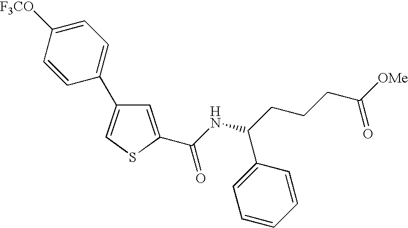

The product (21.3 mg) is obtained according to the process of Example 1, using the product of Preparation 4 as substrate.

Yield: 15%

¹H NMR (DMSO) δ (Ppm): 8.90 (m, 1H), 8.40 (s, 1H), 8.10 (s, 1H), 7.82 (d, 2H), 7.46 (m, 1H), 7.40 (m, 6H), 7.20 (m, 1H), 4.90 (m, 1H), 3.50 (s, 3H), 2.40 (m, 2H), 2.20 (m, 2H)

MS: MH⁺464

HPLC: 100%

### EXAMPLE 14

### (4R)-3-{[4-(4-Trifluoromethoxyphenyl)thien-2-yl]carboxamido}-4-phenylpentanoic acid

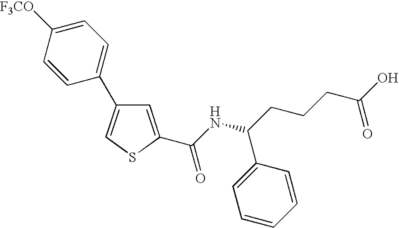

The product (173 mg) is obtained according to the process of Example 2, using the compound obtained in Example 13 as substrate.

Yield: 73%

¹H NMR (DMSO) δ (ppm): 8.90 (m, 1H), 8.40 (s, 1H), 8.10 (s, 1H), 7.82 (d, 2H), 7.46 (m, 2H), 7.36 (m, 5H), 4.90 (m, 1H), 2.40 (m, 2H), 2.20 (m, 2H)

MS: MH⁺450

HPLC: 98.9%

### EXAMPLE 15

### Methyl (2S){[4-(4-Cyclohexylphenyl)thien-2-yl]carboxamido}(phenyl) acetate

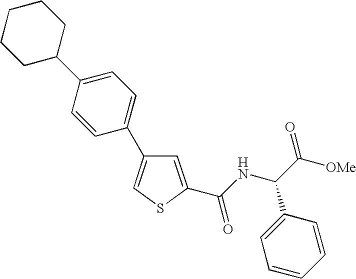

The product (0.155 g) is obtained successively according to the process of stage 1 of Preparation 1, using the product of Preparation 6 and of Preparation 5 as substrate, and then the process of Example 2, using the product obtained in the preceding stage as substrate, and then the process of Example 1, using ethyl (2S)-amino(phenyl)acetate as substrate.

Yield: 55,4%

¹H NMR (DMSO) δ (ppm): 9.20 (m, 1H), 8.40 (s, 1H), 8.10 (s, 1H), 7.70 (d, 2H), 7.46 (m, 5H), 7.30 (m, 2H), 5.60 (d, 1H), 3.90 (s, 3H), 1.80 (m, 5H), 1.40 (m, 6H)

MS: MH⁺434

HPLC: 100%

### EXAMPLE 16

### (2S)-{[4-(4-cyclohexylphenyl)thien-2-yl]carboxamido}(phenyl)acetic acid

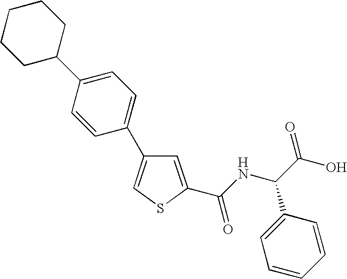

The product (37.4 mg) is obtained according to the process of Example 2, using the compound obtained in Example 15 as substrate.

Yield: 25%

¹H NMR (DMSO) δ (ppm): 12.9 (m, 1H), 9.10 (m, 1H), 8.40 (s, 1H), 8.10 (s, 1H), 7.82 (d, 2H), 7.46 (m, 5H), 7.30 (m, 2H), 5.50 (m, 1H), 1.80 (m, 5H), 1.40 (m, 6H)

MS: MH⁺420

HPLC: 100%

### EXAMPLE 17

### Ethyl (2S)-({4-[4-(4-acetylphenyl)phenyl]thien-2-yl}carboxamido)-(phenyl)acetate

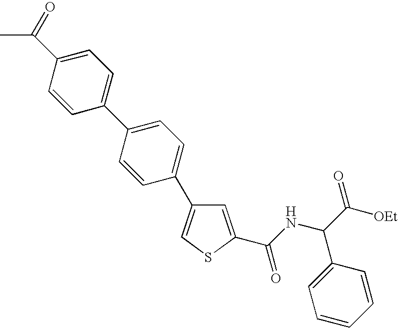 Stage 1: Methyl 4-[4-(4-acetyl-phenyl)phenyl]-thiophene-2-carboxylate

The product (1.73 g) is obtained according to the process of stage 1 of Preparation 1, using the product of Preparation 6 and (4-acetyl-phenyl)boronic acid as substrates.

Yield: 76%

MS: MH⁺337

Stage 2: 4-[4-(4-Acetyl-phenyl)phenyl]-thiophene-2-carboxylic acid

The product (1 g) is obtained according to the process of Example 2, using the product obtained in stage 1 above as substrate.

Yield: 61%

MS: MH⁺323

Stage 3: Ethyl (2S)-({4-[4-(4-Acetyl-phenyl)phenyl]thien-2-yl}carboxamido)-(phenyl)acetate

The product (360 mg) is obtained according to the process of Example 1, using ethyl (2S)-amino(phenyl)acetate hydrochloride as substrate.

Yield: 44%

¹H NMR (CDCl₃) δ (Ppm): 9.20 (d, 1H), 8.60 (s, 1H), 8.25 (s, 1H), 8.05 (d, 2H), 7.90 (d, 2H), 7.85 (s, 4H), 7.50 (m, 2H), 7.40 (m, 3H), 5.60 (d, 1H), 4.20 (m, 2H), 2.65 (s, 3H), 1.20 (t, 3H)

### EXAMPLE 18

### (2S)4-[({4-[4-(4-Acetylphenyl)phenyl]thien-2-yl}carbonyl)amino]-(phenyl)acetic acid

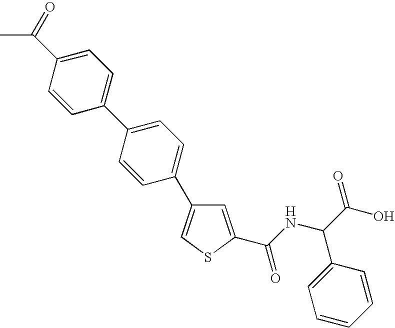

The product (16 mg) is obtained according to the process of Example 2, using the compound obtained in Example 17 as substrate.

Yield: 5%

¹H NMR (DMSO) δ (ppm): 13.0 (bs, 1H), 9.10 (d, 1-H), 8.60 (s, 1H), 8.25 (s, 1H), 8.05 (d, 2H), 7.90 (m, 6H), 7.50 (m, 2H), 7.40 (m, 3H), 5.55 (d, 1H), 2.60 (s, 3H)

MS: MH⁺456

HPLC: 95.9%

### EXAMPLE 19

### Ethyl (2R₃-({4-[4-(4-acetyl-phenyl)phenyl]thien-2-yl}carboxamido) (phenyl)propanoate

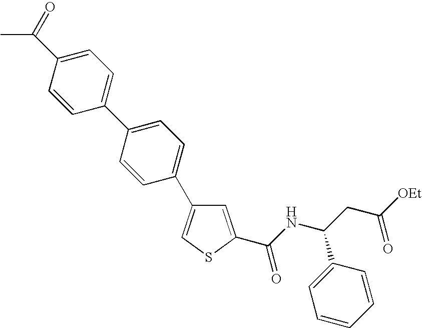

The product (1.012 g) is obtained according to the process of Example 17, replacing the ethyl (2S)-amino(phenyl)acetate hydrochloride with ethyl (2R)-amino(phenyl)propanoate hydrochloride.

Yield: 66%

¹H NMR (DMSO) δ (ppm): 9.10 (d, 1H), 8.30 (s, 1H), 8.25 (s, 1H), 8.15 (d, 2H), 7.90 (m, 6H), 7.35 (m, 5H), 5.40 (m, 1H), 4.10 (q, 2H), 3.0 (m, 2H), 2.60 (s, 3H), 1.30 (t, 3H)

MS: MH⁺498

HPLC: 97.7%

### EXAMPLE 20

### (2R)-3-({1414-(4-Acetylphenyl)phenyl]thien-2-yl}carboxamido) (phenyl)propanoic acid

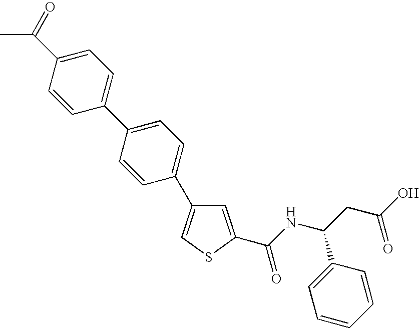

The product (0.641 g) is obtained according to the process of Example 2, using the compound obtained in Example 19 as substrate.

Yield: 91%

¹H NMR (DMSO) δ (ppm): 9.10 (d, 1H), 8.3 (s, 1H), 8.25 (s, 1H), 8.15 (d, 2H), 7.90 (m, 6H), 7.35 (m, 5H), 5.40 (m, 1H), 3.0 (m, 2H), 2.60 (s, 3H)

MS: MH⁺470

HPLC: 100%

### EXAMPLE 21

### Ethyl (2R₃-({4-[4-(pyridyl)phenyl]thien-2-yl}carboxamido)-(phenyl)propanoate

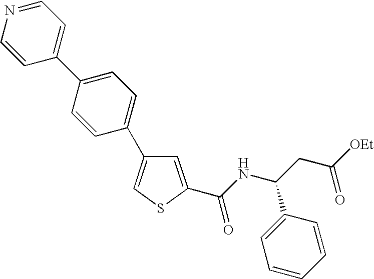 Stage 1: Methyl 4-[4-(pyrid-4-yl)phenyl]thiophene-2-carboxylate hydrochloride

The crude product (60 g) is obtained in the form of a yellow solid by applying the process of stage 1 of Preparation 1, using the product of Preparation 6 and (4-pyridyl)boronic acid as substrates. The organic phase obtained after extraction is acidified with 2M hydrochloric acid solution to precipitate out the desired product, which is finally isolated by filtration.

¹H NMR (DMSO) δ (ppm): 8.91 (d, 2H), 8.50 (s, 1H), 8.38 (s, 1H), 8.36 (d, 2H), 8.10 (d, 2H), 8.06 (d, 2H), 3.45 (s, 3H)

Stage 2: 4-[4-(4-Acetylphenyl)phenyl]thiophene-2-carboxylic acid hydrochloride

The product (19.4 g) is obtained according to the process of Example 2, using the product obtained in stage 1 above as substrate.

Yield for stages 1 and 2: 44.5%

¹H NMR (DMSO) δ (ppm): 8.88 (d, 2H), 8.43 (s, 1H), 8.30 (d, 2H), 8.28 (s, 1H), 8.08 (d, 2H), 8.02 (d, 2H)

Stage 3: Ethyl (2R)-3[(({4-[4-(4-acetylphenyl)phenyl]thien-2-yl}carboxamido)-(phenyl)propanoate

The product (1.43 g) is obtained according to the process of Example 1, using ethyl (2R)-amino(phenyl)propanoate hydrochloride as substrate.

Yield: 100%

¹H NMR (DMSO) δ (ppm): 8.15 (d, 1H), 8.76 (d, 2H), 8.35 (s, 1H), 8.22 (s, 1H), 7.95 (d, 2H), 7.90 (d, 2H), 7.88 (d, 2H), 7.44 (d, 2H), 7.35 (t, 2H), 7.26 (t, 1H), 5.42 (q, 1H), 4.05 (q, 2H), 2.95 (m, 2H), 1.10 (t, 3H)

HPLC: 100%

### EXAMPLE 22

### (2R)-3-({[4-[(Pyrid-4-yl)phenyl]thien-2-yl}carboxamido)(phenyl) propanoic acid hydrochloride

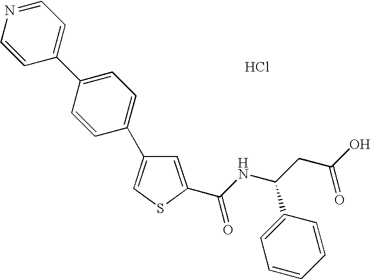

The product (0.990 g) is obtained according to the process of Example 2, using the compound obtained in Example 21 as substrate.

Yield: 68%

¹H NMR (DMSO) δ (ppm): 9.02 (d, 1H), 8.88 (d, 2H), 8.46 (s, 1H), 8.34 (d, 2H), 8.30 (s, 1H), 8.11 (d, 2H), 7.95 (d, 2H), 7.44 (d, 2H), 7.35 (t, 2H), 7.26 (t, 1H), 5.43 (q, 1H), 2.89 (m, 2H)

HPLC: 100%

### EXAMPLE 23

### N-(3-Benzyloxy-1-phenyl-2-oxopropyl)-4-(4-trifluoromethoxyphenyl)thiophene-2-carboxamide

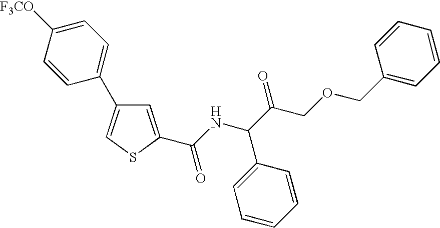

The product (17.8 mg) is obtained according to the process of Example 1, using the compound obtained in Preparation 7 as substrate.

Yield: 23%

¹H NMR (DMSO) δ (ppm): 9.10 (d, 1H), 8.50 (s, 1H), 8.25 (s, 1H), 7.80 (d, 2H), 7.40 (m, 12H), 5.90 (m, 1H), 4.40 (m, 4H)

MS: MH⁺526

HPLC: 100%

### EXAMPLE 24

### Pharmacological Studies on the Compounds of the Invention

in vitro Evaluation of the Inhibitory Activity of the Compounds of the Invention on MMP-12:

The inhibitory activity of the compounds of formula (I) on metalloprotease-12 is evaluated by testing the capacity of the compounds of the invention to inhibit the proteolysis of a peptide that is an MMP-12 substrate.

The substrate peptide used (fluorigenic peptide-1: FP-1) in the test has the following sequence: Mca-Pro-Leu-Gly-Leu-Dap(Dnp)-Ala-Arg-NH₂.

The inhibitory activity of a compound of formula (I) is expressed as the IC₅₀ value, which represents the concentration of inhibitor for which a 50% inhibition of the metalloprotease is observed.

The reaction starts with the sequential addition of 41 μl of FP-1 substrate (final concentration of 10 μM) to a buffer solution of 50 mM of Tris-HCl and 10 mM of CaCl₂, and containing 5 mM of hydroxamic acid and 5 μl of the enzyme diluted in a 0.005% Brij-35 buffer solution. The microplates are incubated for 20 minutes at room temperature. The compounds of the invention are tested at concentrations ranging from 0.3 to 30 μM. The measurement of the amount of proteolysis of the peptide substrate is monitored by means of a measurement of absorbance at 405 nm using a microplate spectrophotometer, at room temperature. The IC₅₀ values are calculated from curves in which the percentage of the catalytic activity relative to the control is represented on the x-axis and the inhibitor concentration is represented on the y-axis.

The test described above for the inhibition of MMP-12 is adapted and used to determine the capacity of the compounds of formula (I) to inhibit the metalloproteases MMP-1, MMP-2, MMP-3, MMP-7, MMP-9, MMP-13 and MMP-14. The results obtained show that the compounds of the invention generally have IC₅₀ values for MMP-12 that are from 5 to more than 100 times lower than the IC₅₀ values obtained for the same compound with the other metalloproteases tested, thus proving their capacity for selective inhibition with respect to metalloprotease-12 (MMP-12). More specifically, the compounds of the present invention generally show selectivity with a factor of greater than 50 towards the metalloproteases mentioned above, except with regard to MMP-13. Thus, the compounds of the present invention also show inhibitory activity on MMP-13, also allowing the use of the pharmaceutical compositions containing one or more compounds of the invention for the treatment of pathologies associated with an activity of MMP-13. Among these pathologies that may be mentioned, as a guide and with no limitation, are cancer, osteoporosis, osteoarthritis, arthritis, rheumatoid arthritis, atherosclerosis, multiple sclerosis, cardiac insufficiency, asthma and chronic obstructive bronchopneumopathy.

By way of example and with no limitation of the invention, the table shows a number of results of activity of the compounds of the invention with respect to MMP-12 and MMP-13.

| | IC₅₀ (μM) | IC₅₀ (μM) |
|---|---|---|
| Example | MMP-12 | MMP-13 |
| 2 | 0.38 | 4.5 |
| 4 | 0.47 | 5.6 |
| 6 | 0.420 | 3.6 |
| 8 | 0.180 | 2.2 |
| 9 | 0.077 | 0.96 |
| 12 | 0.530 | 8.3 |
| 16 | 0.540 | 6.5 |
| 18 | 0.040 | 0.120 |
| 20 | 0.028 | 0.069 |
| 22 | 0.014 | 0.270 |

1. A compound of formula (I): 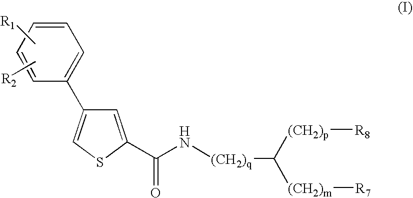
R_{2<}
H(CH₂)ₚ—R₈ I S N(CH₂)_{q} 0 (CH2)ₘ R.
a steroisomer thereof, or a pharmaceutically acceptable salt of said compound or said steroisomer, wherein:
R₁ and R₂ are independently hydrogen, halogen, cyano, nitro, halo(C₁-C₆)alkyl, halo(C₁-C₆)alkoxy, (C₁-C₆)alkyl, (C₂-C₆)alkenyl, (C₂-C₆)alkynyl, —OR₄, —NR₄R₅, —S(O)ₙR₄, —C(O)R₄, —CO₂R₄, —O—C(O)R₄, —C(O)NR₄R₅, —NR₅—C(O)R₄, —NR₅—SO₂R₄, -T-CN, -T-OR₄, -T-OCF₃, -T-NR₄R₅, -T-S(O)ₙR₄, -T-C(O)R₄, -T-CO₂R₄, -T-O—C(O)R₄, -T-C(O)NR₄R₅, -T-NR₅—C(O)R₄, -T-NR₅—SO₂R₄, —R₆, or -T-R₆;
m is 0, 1, 2, 3 or 4;
p is 0, 1, 2, 3 or 4;
q is 0, 1, 2, 3 or 4;
R₇ is aryl, cycloalkyl or heterocycle, optionally and independently substituted with one to three halogen, cyano, nitro, halo(C₁-C₆)alkyl, halo(C₁-C₆)alkoxy, (C₁-C₆)alkyl, hydroxyl, (C₁-C₆)alkoxy, amino, mono(C₁-C₆)alkylamino, di(C₁-C₆)alkylamino, mercapto, (C₁-C₆)alkylthio, (C₁-C₇)acyl, (C₁-C₆)alkyl sulphoxide, carboxyl or (C₁-C₆)alkoxycarbonyl;
R₈ is carboxyl, (C₁-C₆)alkoxycarbonyl, (C₁-C₇)acyl, hydroxy(C₁-C₆)alkylcarbonyl, (C₁-C₆)alkoxy(C₁-C₆)alkylcarbonyl, tetrazole, amino, aminocarbonyl or amino(C₁-C₆)alkylcarbonyl, said amino, aminocarbonyl and amino(C₁-C₆)alkylcarbonyl optionally and independently substituted with one or two (C₁-C₆)alkyl or cycloalkyl;
n is 0, 1 or 2;
T is (C₁-C₆)alkylenyl optionally substituted with oxo, halogen, (C₁-C₆)alkoxy, hydroxyl, amino, mono(C₁-C₆)alkylamino or di(C₁-C₆)alkylamino, and wherein one of the carbon atoms is optionally replaced with O, S, N, or —N(C₁-C₆)alkyl-;
R₄ is H, (C₁-C₆)alkyl, aryl, cycloalkyl or a heterocycle;
R₅ is H or (C₁-C₆)alkyl;
R₆ is aryl, cycloalkyl or heterocyle, optionally and independently substituted with one to five halogen, cyano, nitro, oxo, halo(C₁-C₆)alkyl, halo(C₁-C₆)alkoxy, (C₁-C₆)alkyl, (C₁-C₆)alkenyl, —OR₄₀, —NR₄₀OR₅₀, —S(O)ₙ₁R₄₀, —C(O)R₄₀, —CO₂R₄₀, —O—C(O)R₄₀, —C(O)NR₄₀R₅₀, —NR₅₀—C(O)R₄₀, —NR₅₀-SO₂R₄₀, -T₁-CN, -T₁-OR₄₀, -T, —OCF₃, -T₁-NR₄₀R₅₀, -T₁-S(O)ₙR₄₀, -T₁-C(O)R₄₀, -T₁-CO₂R₄₀, -T₁-O—C(O)R₄₀, -T₁-C(O)NR₄₀R₅₀, -T₁-NR₅₀—C(O)R₄₀or -T₁-NR₅₀—SO₂R₄₀, wherein R₄₀, R₅₀, T₁, and n₁, have the same meanings as R₄, R₅, T and n, respectively, as defined above

2. A compound of claim 1, wherein:
R₁ is halo(C₁-C₆)alkoxy, (C₁-C₆)alkyl, —OR₄, —SR₄ or —R₆;
R₄ is H or (C₁-C₆)alkyl;
R₆ is phenyl, cyclohexyl or a heterocycle, each optionally and independently substituted with one or two halogen, halo(C₁-C₆)alkyl-, halo(C₁-C₆)alkoxy-, (C₁-C₆)alkyl, cyano, -T-CN, —OR₄₀-T-OR₄₀, —NR₄₀R₅₀, —S(O)ₙ₁R₄₀ or —C(O)R₄₀;
R₄₀ is H, (C₁-C₆)alkyl or phenyl;
R₅₀ is H or (C₁-C₆)alkyl;
T, is (C₁-C₆)alkylenyl;
n₁ is 0, 1 or 2;
R₂ is H;
m is 0, 1, 2, 3 or 4;
p is 0, 1, 2, 3 or 4;
q is 0, 1, 2, 3 or 4;
R₇ is phenyl, monocyclic cycloalkyl or a 5- or 6-membered monocyclic heterocycle comprising one or two hetero atoms, which may be identical or different, selected from O or N, optionally and independently substituted with one or two halogen, cyano, nitro, halo(C₁-C₆)alkyl, halo(C₁-C₆)alkoxy, (C₁-C₆)alkyl, hydroxy, (C₁-C₆)alkoxy, amino, mono(C₁-C₆)alkylamino, di(C₁-C₆)alkylamino, mercapto, (C₁-C₆)alkylthio, (C₁-C₇)acyl, (C₁-C₆)alkyl sulphoxide, carboxyl or (C₁-C₆)alkoxycarbonyl;
R₈ is carboxyl, (C₁-C₆)alkoxycarbonyl, aminocarbonyl, (C₁-C₆)alkylcarbonyl, amino, hydroxy(C₁-C₆)alkylcarbonyl, (C₁-C₆)alkoxy(C₁-C₆)alkylcarbonyl or amino(C₁-C₆)alkylcarbonyl, the amino portion in each optionally and independently substituted with one or two (C₁-C₆)alkyl;
or a steroisomer thereof, or a pharmaceutically acceptable salt of said compound or said steroisomer.

3. A compound of claim 1 wherein R₁ is located in the para position, or a steroisomer thereof, or a pharmaceutically acceptable salt of said compound or said steroisomer.

4. A compound of claim 1 wherein R₁ is trifluoromethoxy, 4-acetylphenyl, 4-pyridyl, 3-pyridyl, N-pyrrolidinyl, 1-methylpyrrol-3-yl, 3,6-dihydro-2H-pyrid-1-yl, cyclohexyl, 2-hydroxy-4-pyridyl and 2-hydroxyphenyl, or a steroisomer thereof, or a pharmaceutically acceptable salt of said compound or said steroisomer.

5. A compound of claim 4 wherein R₁ is trifluoromethoxy, 4-acetylphenyl, cyclohexyl and 4-pyridyl, or a steroisomer thereof, or a pharmaceutically acceptable salt of said compound or said steroisomer.

6. A compound of claim 1 wherein q is 0, or a steroisomer thereof, or a pharmaceutically acceptable salt of said compound or said steroisomer.

7. A compound of claim 1 wherein m is 0 or 1, or a steroisomer thereof, or a pharmaceutically acceptable salt of said compound or said steroisomer.

8. A compound of claim 1 wherein p is 0 or 1, or a steroisomer thereof, or a pharmaceutically acceptable salt of said compound or said steroisomer.

9. A compound of claim 1 wherein R₇ is phenyl, or a steroisomer thereof, or a pharmaceutically acceptable salt of said compound or said steroisomer.

10. A compound of claim 1 wherein R₈ is carboxyl or aminocarbonyl, or a steroisomer thereof, or a pharmaceutically acceptable salt of said compound or said steroisomer.

11. A compound selected from:
ethyl (2S)-{[4-(4-trifluoromethoxyphenyl)thien-2-yl]carboxamido}(phenyl)acetate;
(2S)-({[4-(4-trifluoromethoxyphenyl)thien-2-yl]carboxamido}(phenyl)acetic acid;
ethyl (2R)-{[4-(4-trifluoromethoxyphenyl)thien-2-yl]carboxamido}(phenyl)acetate;
(2R)-{[4-(4-trifluoromethoxyphenyl)thien-2-yl]carboxamido}(phenyl)acetic acid;
ethyl 3-{[4-(4-trifluoromethoxyphenyl)thien-2-yl]carboxamido)-3-phenyl-propanoate;
{[4-(4-trifluoromethoxyphenyl)thien-2-yl]carboxamido}(phenyl)propanoic acid;
(2S)-{[4-(4-trifluoromethoxyphenyl)thien-2-yl]carboxamido}(phenyl)propanoic acid;
(2R)-{[4-(4-Trifluoromethoxyphenyl)thien-2-yl]carboxamido}(phenyl)propanoic acid;
N-(3-amino-3-oxo-1-phenylpropyl)-4-(4-trifluoromethoxyphenyl)thiophene-2-carboxamide;
4-(4-trifluoromethoxyphenyl)-N-[(2S)-2-(cyclohexylamino)-2-phenylethanoyl]-thiophene-2-carboxamide;
ethyl (3S)-3-{[4-(4-trifluoromethoxyphenyl)thien-2-yl]carboxamido}-4-phenylbutanoate;
(3S)-3-{[4-(4-trifluoromethoxyphenyl)thien-2-yl]carboxamido)-4-phenylbutanoic acid;
methyl (4R)-3-{[4-(4-Trifluoromethoxyphenyl)thien-2-yl]carboxamido}-4-phenylpentanoate;
(4R)-3-{[4-(4-trifluoromethoxyphenyl)thien-2-yl]carboxamido}-4-phenylpentanoic acid;
methyl (2S)-{[4-(4-cyclohexylphenyl)thien-2-yl]carboxamido} (phenyl)acetate;
(2S)-{[4-(4-cyclohexylphenyl)thien-2-yl]carboxamido}(phenyl)acetic acid;
ethyl (2S)-({4-[4-(4-acetylphenyl)phenyl]thien-2-yl}carboxamido)(phenyl)acetate;
(2S)-4-[({4-[4-(4-acetylphenyl)phenyl]thien-2-yl}carbonyl)amino](phenyl)acetic acid;
ethyl (2R)-3-({4-[4-(4-Acetyl-phenyl)phenyl]thien-2-yl}carboxamido)(phenyl)-propanoate;
(2R)-3-({4-[4-(4-acetyl-phenyl)phenyl]thien-2-yl}carboxamido)(phenyl)propanoic acid;
ethyl (2R)-3-({4-[4-(pyrid-4-yl)phenyl]thien-2-yl}carboxamido)(phenyl)propanoate;
(2R)-3-({[4-[(pyrid-4-yl)phenyl]-thien-2-yl}carboxamido)(phenyl)propanoic acid hydrochloride; or
N-(3-benzyloxy-1-phenyl-2-oxopropyl)-4-(4-trifluoromethoxyphenyl)thiophene-2-carboxamide, or
a steroisomer thereof, or a pharmaceutically acceptable salt of said compound or said steroisomer.

12. A pharmaceutical composition comprising a compound of claim 1, a stereoisomer thereof, or a pharmaceutically acceptable salt of said compound or stereoisomer, and a pharmaceutically acceptable carrier, vehicle or dilluent.

13. A method of treating pathologies requiring the action of a metalloprotease-12 or a metalloprotease-13 inhibitor in a mammal comprising administering to said mammal in need of such treatment a therapeutically effective amount of a compound of claim 1, a stereoisomer thereof, or a pharmaceutically acceptable salt of said compound or said stereoisomer, or a pharmaceutical composition comprising a compound of claim 1, a stereoisomer thereof, or a pharmaceutically acceptable salt of said compound or stereoisomer, and a pharmaceutically acceptable carrier, vehicle or dilluent.

14. A method of claim 13 wherein said pathology requires the action of a metalloprotease-12 inhibitor.

15. A method of treating chronic obstructive bronchopneumopathy (COPD), emphysema, chronic bronchitis, chronic pulmonary inflammation, asthma, mucoviscidosis, acute respiratory distress syndrome (ARDS), respiratory allergies including allergic rhinitis, and also diseases associated with the production of TNFα, including severe fibrotic pulmonary disease, pulmonary sarcoidosis or silicosis in a mammal comprising administering to said mammal in need of such treatment a therapeutically effective amount of a compound of claim 1, or a stereoisomer thereof, or a pharmaceutically acceptable salt of said compound or said stereoisomer, or a pharmaceutical composition comprising a compound of claim 1, a stereoisomer thereof, or a pharmaceutically acceptable salt of said compound or stereoisomer, and a pharmaceutically acceptable carrier, vehicle or dilluent.

16. A method of treating cancer, osteoporosis, osteoarthritis, arthritis, rheumatoid arthritis, atherosclerosis, multiple sclerosis or cardiac insufficiency in a mammal comprising administering to said mammal in need of such treatment a therapeutically effective amount of a compound of claim 1, or a stereoisomer thereof, or a pharmaceutically acceptable salt of said compound or said stereoisomer, or a pharmaceutical composition comprising a compound of claim 1, a stereoisomer thereof, or a pharmaceutically acceptable salt of said compound or stereoisomer, and a pharmaceutically acceptable carrier, vehicle or dilluent.

17. A method of treating chronic obstructive bronchopneumopathy, emphysema or chronic bronchitis in a mammal comprising administering to said mammal in need of such treatment a therapeutically effective amount of a compound of claim 1, or a stereoisomer thereof, or a pharmaceutically acceptable salt of said compound or said, stereoisomer, or a pharmaceutical composition comprising a compound of claim 1, a stereoisomer thereof, or a pharmaceutically acceptable salt of said compound or stereoisomer, and a pharmaceutically acceptable carrier, vehicle or dilluent.

18. A method of treating tobacco-related emphysema in a mammal comprising administering to said mammal in need of such treatment a therapeutically effective amount of a compound of claim 1, or a stereoisomer thereof, or a pharmaceutically acceptable salt of said compound or said stereoisomer, or a pharmaceutical composition comprising a compound of claim 1, a stereoisomer thereof, or a pharmaceutically acceptable salt of said compound or stereoisomer, and a pharmaceutically acceptable carrier, vehicle or dilluent.

19. A method of treating asthma in a mammal comprising administering to said mammal in need of such treatment a therapeutically effective amount of a compound of claim 1, or a stereoisomer thereof, or a pharmaceutically acceptable salt of said compound or said stereoisomer, or a pharmaceutical composition comprising a compound of claim 1, a stereoisomer thereof, or a pharmaceutically acceptable salt of said compound or stereoisomer, and a pharmaceutically acceptable carrier, vehicle or dilluent.
